(19) United States
(12) Patent Application Publication
(10) Pub. No.: US 2023/0227763 A1
(43) Pub. Date: Jul. 20, 2023

(54) Device for Growing Microorganisms

(71) Applicant: 3M INNOVATIVE PROPERTIES COMPANY, Saint Paul, MN (US)

(72) Inventors: Alexi J. Young, Lansing, MI (US); Kurt Jonathan Halverson, Lansing, MI (US); Steven P. Swanson, Lansing, MI (US); Evan Douglas Brutinel, Lansing, MI (US); Caleb Timothy Nelson, Lansing, MI (US)

(73) Assignee: NEOGEN FOOD SAFETY US HOLDCO CORPORATION, LANSING, MI (US)

(21) Appl. No.: 18/002,468

(22) PCT Filed: Jun. 10, 2021
(86) PCT No.: PCT/IB2021/055122

§ 371 Date: Dec. 19, 2022

Related U.S. Application Data
(60) Provisional application No. 62/705,275, filed on Jun. 19, 2020.
(60) Provisional application No. 63/185,645, filed on May 7, 2021.

(51) Int. Cl. C12M 1/12; C12M 1/00

(52) U.S. Cl. CPC C12M 25/06; C12M 23/04; C12M 23/20; C12M 23/22; C12M 23/38

(57) ABSTRACT

A device for growing microorganisms. The device can include a self-supporting, water-proof substrate having an inner-facing surface and an outer-facing surface; a cover sheet having an inner-facing surface and an outer-facing surface, the cover sheet adhered to at least a portion of the substrate; a microstructured surface on the inner-facing surface of the coversheet, wherein the microstructured surface comprises a plurality of microstructures; a substantially dry, gelling agent and a substantially dry, microbial growth nutrient composition disposed on the microstructured surface of the microstructured film or on a portion of the inner-facing surface of the substrate.

### BACKGROUND

A wide variety of culture devices have been developed. As one example, culture devices have been developed by 3M Company (hereafter “3M”) of St. Paul, Minn. In particular, culture devices are sold by 3M under the trade name PETRIFILM plates. Culture devices can be utilized to facilitate the rapid growth and detection of microorganisms commonly associated with food contamination, including, for example, aerobic bacteria, E. coli, coliforms, enterobacteria, yeast, mold, Staphylococcus aureus, Listeria, Campylobacter, and the like. The use of PETRIFILM plates, or other growth media, can simplify bacterial testing of food samples, for instance.

Culture devices can be used to enumerate or identify the presence of bacteria so that corrective measures can be performed (in the case of food testing) or proper diagnosis can be made (in the case of medical use). In other applications, culture devices may be used to rapidly grow microorganisms in laboratory samples, e.g., for experimental purposes.

### SUMMARY

PETRIFILM plates are inoculated with sample by first lifting the cover film, pipetting a known volume (typically 1 milliliter) of test sample centered on the bottom film, slowly lowering the top film to contact the sample liquid, and finally spreading the liquid by pressing on the top film with a circular device. Care must be taken during the spreading step to provide uniform pressure over the sample to avoid uneven distribution of the sample. After spreading the films must be left to stand for several minutes to hydrate the dried gelling agents.

The device of the current application can provide advantages: it can be self-spreading, the device can be moved immediately after inoculation without leaking sample, and pressure (such as stacking) can be placed onto the newly inoculated sample without leaking the sample out of the inoculated area. Self-spreading means that once the aqueous sample is added to the device, the sample distributes uniformly over a controlled area without any subsequent user intervention.

When the liquid sample is added to the device, the liquid can spread to the outer edge or edges of the surface without the need to apply external pressure to the cover sheet of the device.

Thus, in one aspect, the present disclosure provides a device for growing microorganisms. The device can include a self-supporting, water-proof substrate having an inner-facing surface and an outer-facing surface; a cover sheet having an inner-facing surface and an outer-facing surface, the cover sheet adhered to at least a portion of the substrate; a microstructured surface on the inner-facing surface of the coversheet, wherein the microstructured surface comprises a plurality of microstructures; a substantially dry, gelling agent and a substantially dry, microbial growth nutrient composition disposed on the microstructured surface of the microstructured film or on a portion of the inner-facing surface of the substrate.

In another aspect, the present disclosure provides a device for growing microorganisms. The device can include a self-supporting, water-proof substrate having an inner-facing surface and an outer-facing surface; a cover sheet having an inner-facing surface and an outer-facing surface, the cover sheet adhered to at least a portion of the substrate; a microstructured surface on the inner-facing surface of the substrate, wherein the microstructured surface comprises a plurality of microstructures; a substantially dry, gelling agent and a substantially dry, microbial growth nutrient composition disposed on the microstructured surface of the microstructured film or on a portion of the inner-facing surface of the cover sheet.

In another aspect, the present disclosure provides a method. The method can include providing the device of the current application; adding a predetermined volume of a sample containing at least one microorganism onto the self-supporting, water-proof substrate or the microstructured surface of the microstructured film to form an inoculated device; contacting the cover sheet to the self-supporting, water-proof substrate; incubating the inoculated device; and detecting the presence or an absence of a colony of the target microorganism in the device.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### Definitions

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means+/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

The terms “a”, “an”, and “the” include plural referents unless the content clearly dictates otherwise. Thus, for example, reference to a material containing “a compound” includes a mixture of two or more compounds.

### BRIEF DESCRIPTION OF THE DRAWINGS

The disclosure may be more completely understood in consideration of the following detailed description of various embodiments of the disclosure in connection with the accompanying figures, in which:

FIG. 1 is a perspective view of an exemplary device according to the present disclosure.

FIG. 2 is a perspective view of yet another exemplary device according to the present disclosure.

FIG. 3 is a schematic illustration of a microstructured surface of the present disclosure with a quantity of fluid thereon.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

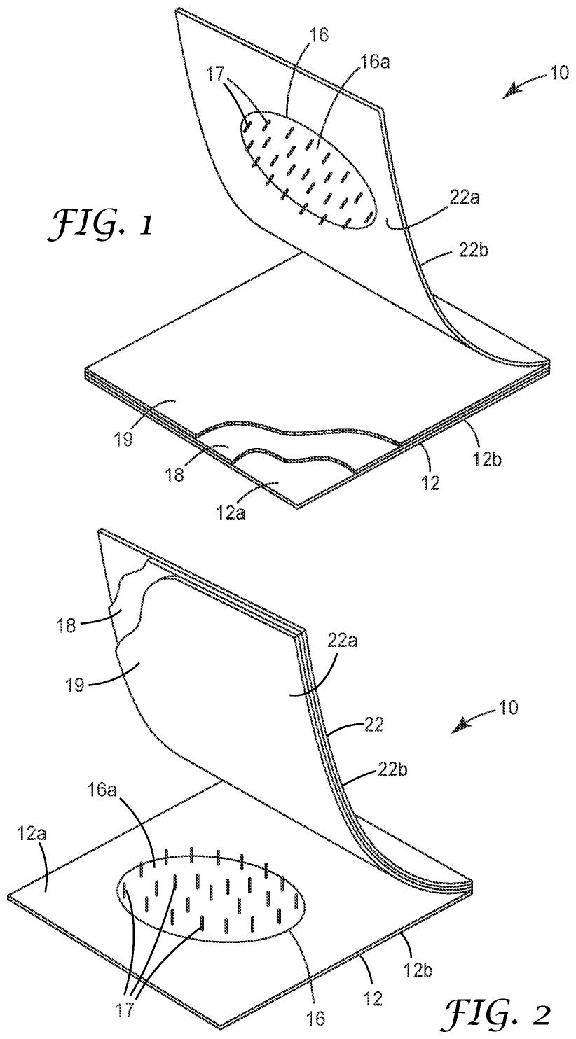

FIG. 1 illustrates an exemplary embodiment of a device for growing microorganisms. The device 10 includes a substrate 12 having a first major surface 12a (e.g., inner-facing surface) and a second major surface 12b (e.g., outer-facing surface) and a cover sheet 22 attached to at least a portion of the substrate 12, where the cover sheet 22 includes a first major surface 22a (e.g., inner-facing surface) facing the substrate 12 and a second major surface 22b (e.g., outer-facing surface). A microstructured film 16 is disposed on the inner-facing surface 22a of the coversheet 22. The microstructured film 16 comprises a microstructured surface 16a having a plurality of microstructures 17. The device 10 further includes a substantially dry, gelling agent and a substantially dry, first microbial growth nutrient composition 18. In the embodiment of FIG. 1, the substantially dry, gelling agent and substantially dry, first microbial growth nutrient composition 18 is disposed on a portion of the inner-facing surface 12a of the substrate 12. Alternatively, the substantially dry, gelling agent 19 and substantially dry, first microbial growth nutrient composition 18 can be disposed on the microstructured surface 16a of the microstructured film 16.

FIG. 2 illustrates an exemplary embodiment of a device for growing microorganisms. The device 10 includes a substrate 12 having a first major surface 12a (e.g., inner-facing surface) and a second major surface 12b (e.g., outer-facing surface) and a cover sheet 22 attached to at least a portion of the substrate 12, where the cover sheet 22 includes a first major surface 22a (e.g., inner-facing surface) facing the substrate 12 and a second major surface 22b (e.g., outer-facing surface). A microstructured film 16 is disposed on the inner-facing surface 12a of the substrate 12. The microstructured film 16 comprises a microstructured surface 16a having a plurality of microstructures 17. The device 10 further includes a substantially dry, gelling agent and a substantially dry, first microbial growth nutrient composition 18. In the embodiment of FIG. 2, the substantially dry, gelling agent and substantially dry, first microbial growth nutrient composition 18 is disposed on a portion of the inner-facing surface 22a of the cover sheet 22. Alternatively, the substantially dry, gelling agent and substantially dry, first microbial growth nutrient composition 18 can be disposed on the microstructured surface 16a of the microstructured film 16.

The device 10 of FIGS. 1 and 2 can further include an adhesive layer between the substantially dry, gelling agent 19 and substantially dry, first microbial growth nutrient composition 18 and the gelling agent 19 can be powder coated onto the adhesive layer. In the embodiments of FIGS. 1 and 2, the microstructured film 16 only covers a part of the inner-facing surface of the coversheet or substrate. In some embodiments, the microstructured film 16 covers at least 20%, 30%, 40%, 50%, 60%, 70%, 80% or 90% of the inner-facing surface of the coversheet or substrate. In some embodiments, the microstructured film 16 covers the whole (100%) inner-facing surface of the coversheet or substrate.

The cross-section of the microstructured surface of the microstructured film 16 can be any shape. Non-limiting examples of useful shapes include a square, a rectangle, a circle, an oval, an ellipse, a polygon, pentagon, a hexagon, a heptagon, and an octagon. The area of the microstructured film 16 may be selected based on, for example, the volume of sample (e.g., aqueous liquid) to be deposited in the microstructured film 16. In any embodiment, for a 0.5-3 milliliter sample, the area of the microstructured film sample-receiving zone is about 10 cm² or about 15 cm². In any embodiment, for a 1-5 milliliter volume of sample, the area of the microstructured film 16 is about 18 cm², 20 cm², about 25 cm², about 30 cm², about 31 cm², or about 25-35 cm².

In some embodiments, the microstructured film 16 can be surface treated to increase the surface energy and to become hydrophilic. Hydrophilicity of the microstructured film 16 can be achieved through one or more of material selection, additives included in the material, or surface treatment. In some embodiments, the microstructured film 16 can have a surface including a surfactant, a surface treatment, a hydrophilic polymer, or a combination thereof. Suitable surfactants include for instance and without limitation, C8-C18 alkane sulfonates; C8-C18 secondary alkane sulfonates; alkylbenzene sulfonates; C8-C18 alkyl sulfates; alkylether sulfates; sodium laureth 4 sulfate; sodium laureth 8 sulfate; dioctylsulfosuccinate, sodium salt; lauroyl lactylate; stearoyl lactylate; or any combination thereof. One or more surfactants can be applied by conventional methods, such as by wiping a coating of the surfactant on the surface of the microstructured film 16 and allowing the coating to dry. A suitable surface treatment includes a hydrophilic coating comprising plasma deposited silicon/oxygen materials and/or diamond-like glass (DLG) materials. Plasma deposition of each of silicon/oxygen materials and DLG material is described, for instance, in PCT Publication No. WO 2007/075665 (Somasiri et al.). Further, examples of suitable DLG materials are disclosed in U.S. Pat. No. 6,696,157 (David et al.), U.S. Pat. No. 6,881,538 (Haddad et al.), and U.S. Pat. No. 8,664,323 (Iyer et al.). Suitable hydrophilic polymers include for instance and without limitation, a polyester, a polyamide, a polyurethane, a poly(vinyl alcohol), a poly(alkylene glycol), a poly(alkylene oxide), a poly(vinyl pyrrolidone), a rubber elastomer, or any combination thereof. In some embodiments, the microstructured film 16 can be surface treated more than once, for example, a first surface plasma treatment followed by a second surface plasma treatment. In some embodiments, surface treatment can include hydrophilic surface treatment covalently bonded to at least a portion of the surface of the microstructured film. In some other embodiments, the microstructured film can have a noncovalent hydrophilic surface treatment, for example, surfactant treatment, disposed to a least a portion of the surface of the microstructured film. The microstructured film can exhibit a capillary rise percent recovery of at least 10%. Typically, the hydrophilic surface treatment includes functional groups selected from a non-zwitterionic sulfonate, a non-zwitterionic carboxylate, a zwitterionic sulfonate, a zwitterionic carboxylate, a zwitterionic phosphate, a zwitterionic phosphonic acid, a zwitterionic phosphonate, or a combination thereof.

In some embodiments, a thin, silicon containing film layer can be applied to the microstructured film 16. The microstructured film 16 can be made by any suitable method, for example, the method described in US 2019/0001326 A1 (Brutinel et al.). In some embodiments, the microstructured film is transparent.

The substrate is water-proof, and is optionally a self-supporting water-proof substrate. In some embodiments, the substrate is a film of a material such as polyester, polypropylene, silicone, or polystyrene, which will not absorb or otherwise be affected by water. Polyester films and polypropylene films having a thickness from about 20 micrometers to about 250 micrometers, as well as polystyrene films having a thickness of about 380 micrometers, have each been found to be suitable for the substrate. Other suitable substrates include paper with a polyethylene or other water-proof coating. An example of a suitable polyethylene-coated paper substrate is “Schoeller Type MIL” photoprint paper (commercially available from Schoeller Pulaski, New York). The substrate may be either transparent or opaque, depending on whether one wishes to view bacterial colonies through the substrate. In some embodiments, the substrate has a square grid pattern printed on the second major surface 12b or 22b to facilitate the counting of bacterial colonies.

The substantially dry, microbial growth nutrient composition can include the microbial growth nutrient composition at a coating weight of 2 milligrams per square inch or more (mg/in²), 5 mg/in² or more, 10 mg/in² or more, 12 mg/in² or more, or 15 mg/in² or more; and at a coating weight of 50 mg/in² or less, 45 mg/in² or less, 40 mg/in² or less, 35 mg/in² or less, 30 mg/in² or less, 24 mg/in² or less, 22 mg/in² or less, 20 mg/in² or less, or 18 mg/in² or less. One suitable method for applying the microbial growth nutrient composition on the substrate includes preparing an aqueous solution or a suspension including at least the microbial growth nutrient composition, disposing a coating of the solution or suspension on the substrate surface, and drying the coating to form the substantially dry microbial growth nutrient composition. The skilled practitioner is capable of selecting a suitable coating method, including for instance and without limitation, knife-coating, gravure coating, curtain coating, air knife coating spray coating, die coating, draw bar coating, curtain coating or roll-coating. The coating is optionally dried at an elevated temperature (e.g., in a range from 50° C. to 100° C.) or in ambient conditions. In some embodiments, the dried microbial growth nutrient composition contains 75% by weight or more microbial growth nutrients, or 80% by weight or more, or 85% by weight or more, or 90% by weight or more, or 95% by weight or more microbial growth nutrients. Advantageously, in certain embodiments a greater amount of microbial growth nutrients can be included in the device than in devices in which the microbial growth nutrient composition is powder coated to an adhesive layer and/or combined with a substantial amount of a cold-water-soluble gelling agent.

The adhesive layer can have an adhesive composition. The adhesive composition can be (substantially) water-insoluble and non-inhibitory to the growth of microorganisms. In some embodiments, the adhesive composition is sufficiently transparent when wet to enable the viewing of bacterial colonies through the film coated with the adhesive. In some embodiments, the adhesive composition can be a pressure-sensitive adhesive. In some other embodiments, heat-activated adhesives in which a lower melting substance is coated onto a higher melting substance may also be used. Water-activated adhesives such as mucilage may also be useful.

Suitable adhesives are transparent when wetted with water. As noted above, the adhesive composition is often water insoluble. In certain embodiments, the adhesive composition comprises a solvent based adhesive. The first adhesive composition and, if present, second adhesive composition often is a pressure sensitive adhesive. For instance, the adhesive may be a pressure-sensitive adhesive such as a water-insoluble adhesive comprising a copolymer of an alkyl acrylate monomer and an alkyl amide monomer or a copolymer of an alkyl acrylate monomer and an acrylic acid. Preferably the weight ratio of alkyl acrylate monomer to alkyl amide monomer in these copolymers is from about 90:10 to 99:1, more preferably 94:6 to 98:2. The alkyl acrylate monomer comprises a lower alkyl (C2 to C10) monomer of acrylic acid, including, for example, isooctyl acrylate (IOA), 2-ethylhexyl acrylate, butyl acrylate, ethyl acrylate, isoamyl acrylate, and mixtures thereof, while the alkyl amide monomer can comprise, without limitation, acrylamide (ACM), methacrylamide, N-vinylpyrrolidone (NVP), N-vinylcaprolactam (NVCL), N-vinyl-2-piperidine, N-(mono- or di-lower alkyl (C2 to C5))(meth)acrylamides, N-methyl(meth)acrylamide, N,N-dimethyl(meth) acrylamides, or mixtures thereof. Suitable adhesives may also include those described in U.S. Pat. Nos. 4,565,783, 5,089,413, 5,681,712, and 5,232,838. In some embodiments, silicone pressure sensitive adhesives may be used, including for example those described in U.S. Pat. Nos. 7,695,818 and 7,371,464.

In the present disclosure, the cover sheet is usually selected to be transparent, in order to facilitate counting of microbial colonies, and is typically also selected to be impermeable to bacteria and have low moisture vapor transmission rate (i.e., the cover sheet prevents undesired contamination of the dehydrated medium during shipping, storage and use of the devices and provides an environment which will support the growth of microorganisms during the incubation period). In some embodiments, the cover sheet has the same properties (e.g., being water-proof) as the substrate. The cover sheet can be selected to provide the amount of oxygen transmission necessary for the type of microorganism desired to be grown. For example, some polyester films have low oxygen permeability (less than 5 g/645 cm²/24 hours per 25 micrometers of thickness) and would be suitable for growing anaerobic bacteria. On the other hand, some polyethylenes have high oxygen permeability (e.g., approximately 500 g/645 cm²/24 hours per 25 micrometers of thickness) and would be suitable for aerobic organisms. Suitable material for the cover sheet includes polypropylene, polyester, polyethylene, polystyrene, or silicone. In certain embodiments, the cover sheet comprises oriented polypropylene, such as biaxially oriented polypropylene, which in some exemplary embodiments has a thickness of about 40 micrometers.

In certain embodiments, the substantially dry, gelling agent contains one or more organic cold-water-soluble agents, such as alginate, carboxymethyl cellulose, tara gum, hydroxyethyl cellulose, hydroxypropyl methylcellulose, guar gum, locust bean gum, xanthan gum, polyacrylamide, polyurethane, polyethylene oxides. Combinations of natural and/or synthetic gelling agents are contemplated. Preferred gelling agents include guar gum, xanthan gum, and locust bean gum, these gelling agents being useful individually or, in any embodiment, in combination with one another. A uniform monolayer of a cold-water-soluble hydrogel-forming composition is desired with sufficient surface area exposed for hydration. Optionally, the powdered cold-water-soluble hydrogel-forming composition may further comprise an inducer, and indicator agent, or a combination of these.

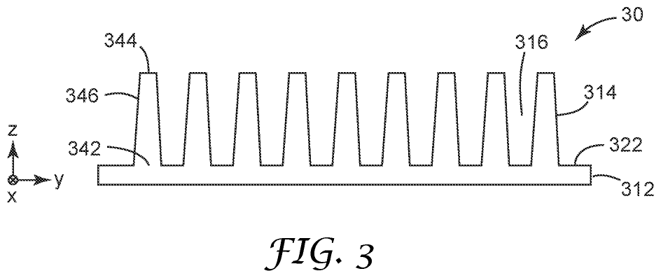

Referring more specifically to FIG. 3, the microstructures 314 each have a conical shape that is generally circular in cross section. It is to be understood that the microstructures 314 may have various cross sectional shapes such as, for example, a circular shape, an oval shape, a square shape, a polygon shape such as a hexagon, etc. The microstructures 314 can be slightly tapered to a smaller cross sectional area adjacent the second end 344 than at the first end 342.

The microstructures 314 have a height “H” which is a longitudinal distance between the first end 342 (the base of microstructures 314) and the second end 344 (the tip of microstructures 314) of the respective microstructures 314. The first and second ends 342 and 344 have a first end width “W1” and a second end width “W2”, respectively. The first width “W1” and the second end width “W2” are representative lateral dimensions of the cross sections of the microstructures 314 in the respective lateral planes. The microstructures 314 each have a tapered shape so that W1 is greater than the corresponding W2. The height “H” of the microstructures 314 can be, for example, no less than 10 microns, no less than 20 microns, no less than 50 microns, or no less than 100 microns. The height of the microstructures 314 can be, for example, no greater than 2 mm, no greater than 1 mm, no greater than 800 microns, or no greater than 500 microns. The average end width (W1+W2)/2 can be, for example, no less than 5 microns, no less than 10 microns, no less than 20 microns, or no less than 50 microns. The average end width (W1+W2)/2 can be, for example, no greater than 1 mm, no greater than 500 microns, no greater than 300 microns, or no greater than 200 microns.

An aspect ratio of the microstructures 314 can be defined as a ratio between an average longitudinal dimension (e.g., along the direction generally perpendicular to the film 310) and an average lateral dimension (e.g., along a lateral, in plane direction generally parallel to the film 310). The microstructures 314 have an aspect ratio that can be defined by H/((W1+W2)/2). In some embodiments, the aspect ratio H/((W1+W2)/2) can be, for example, 0.5 or more, 1 or more, or 2 or more. In some embodiments, the aspect ratio H/((W1+W2)/2) can be, for example, 10 or less, 8 or less, or 6 or less. In some embodiments, the aspect ratio H/((W1+W2)/2) can be between 0.5 and 6. In some embodiments, the aspect ratio H/((W1+W2)/2) can be from 1:1 to 200:1.

The array of microstructures 314 are arranged in two dimensions with columns and rows on the base 312. The microstructures 314 are discrete and separated with each other by continuous cavities 316 therebetween. A pin density of the microstructures 314 is defined as the number of discrete microstructures per area on the base 312. In some embodiments, the pin density can be 50 pins/inch (ppi) or more, 100 ppi or more, 500 ppi or more, or 1000 ppi or more. The pin density can be 20,000 ppi or less, 10,000 ppi or less, 5000 ppi or less, or 3000 ppi or less. In some embodiments, the pin density can be between 100 and 10,000 ppi.

In some embodiments, the plurality of microstructures 314 can be orderly arranged on the microstructured surface of the microstructured film as illustrated in FIG. 3. Here, orderly arranged means that there is a pattern to the placement of the microstructures in relation to each other that is repeated throughout the surface. There may also be multiple different orderly arrangements on the same surface to create a second level of orderly arrangement. In some embodiments, the plurality of microstructures can be randomly arranged on the microstructured surface of the microstructured film. Here, randomly arranged means there is no intentional pattern to the placement of the microstructures relative to each other.

In some embodiments, the inner-facing surface of the cover sheet or the inner-facing surface of the substrate has a microstructured surface with the microstructured surface having a plurality of microstructures. The microstructured surface can partially or completely cover the inner-facing surface of the cover sheet or the substrate. In some embodiments, the microstructured surface is at least partially surrounded by a portion of the cover sheet surface or the substrate surface that is not microstructured (i.e. does not contain microstructure features). The shapes, dimensions, arrangements, and spacings of the microstructures are the same as described above.

In some embodiments, the microstructures are posts. The posts can be in the shape of cylinders such as right-circular cylinders and elliptical cylinders. Post shapes include conical pyramid, trigonal pyramid, and square pyramid shapes. Pyramidal shaped posts can be frustoconical.

The posts can be tapered or non-tapered. Tapered posts can have a wider base than tip. The posts can have a flat or rounded surface at the tip of the post.

Posts can have a circular, elliptical, or oval cross-sectional shape. Posts can have a polygon cross-sectional shape. The cross-sectional shape of a post can be for example a triangle, a square, a rectangle, a trapezoid, a pentagon, a hexagon, a heptagon, an octagon, etc.

In some embodiments, the plurality of posts can be arranged in uniformly spaced rows. When arranged in rows, the posts of adjacent rows can be spaced to be aligned or to be offset.

A method of detecting and enumerating at least one microorganism in a sample is provided. The method includes providing a device according to the current disclosure, adding a predetermined volume of a sample containing at least one microorganism onto the self-supporting, water-proof substrate or the microstructured surface of the microstructured film to form an inoculated device, contacting the cover sheet to the substrate, incubating the inoculated device, and detecting the presence or an absence of a colony of the target microorganism in the device. The gelling agent and growth nutrient composition can be hydrated and form a hydrogel when an aqueous sample is placed into the device, and the hydrogel can partially or completely fills the spaces between the plurality of microstructures.

In some embodiments, the microstructured surface forms the sample receiving zone of the device and a predetermined volume of sample is added to the sample receiving zone. When the liquid sample is added to this sample receiving zone, the liquid can spread to the outer edge or edges of the microstructured surface without the need to apply external pressure to the cover sheet of the device.

The method can further comprise a step of lifting the cover sheet to add the sample. The method can further comprise a step of incubating the device for a period of time at a temperature that facilitates growth and detection of a target microorganism. A person having ordinary skill in the art will recognize the incubation temperature and period of time will depend upon a number of factors (e.g., the target microorganism, nutrients present in the sample, nutrients present in the device, inhibitory agents present in the sample and/or the device) and will adjust the incubation time and temperature accordingly.

The method further comprises a step of detecting a presence or an absence of a colony of the target microorganism in the device. In any embodiment, detecting a presence or an absence of a colony of the target microorganism in the device can comprise detecting a colony (e.g., visually or using machine vision) in the first compartment of the device. In any embodiment, detecting a presence or an absence of a colony of the target microorganism in the device can comprise detecting a change associated with the indicator reagent. The indicator reagent may change from a first state (e.g., substantially colorless or nonfluorescent) to a second state (e.g., colored or fluorescent) in and/or surrounding a colony of the target microorganism. In any embodiment, the colonies can be enumerated and, optionally, the number of colonies of target microorganisms can be recorded. In some embodiments, the microorganisms can be counted using an automated system, such as an automated colony counter.

The device of the current application can provide advantages: it can be self-spreading, the device can be moved immediately after inoculation without leaking sample, and pressure (such as stacking) can be placed onto the newly inoculated sample without leaking the sample out of the inoculated area. Self-spreading means that once the aqueous sample is added to the device, the sample distributes uniformly over a controlled area. The size and shape of the controlled area depends on a number of factors including size, shape, and arrangement of the microstructures as well as the amount of powder cold-water soluble gelling agent present opposite the microstructures.

### EXAMPLES

TABLE 1
Materials

| Material Name/Description | Source |
|---|---|
| BACTO Tryptic Soy Broth (TSB) | Becton, Dickinson and Company, |
| | Franklin Lakes, NJ |
| Butterfield's Buffer | 3M Corporation, Maplewood, MN |
| Guar Gum (Meyprogat 150) | Danisco, Copenhagen, Denmark |
| 2,3,5-Triphenyl Tetrazolium Chloride (TTC) | MilliporeSigma Company, St. Louis, MO |
| 6-Chloro-3-indoxyl-beta-D-galactoside | Biosynth International Inc., Itasca, IL |
| 5-Bromo-4-chloro-3-indoxyl-beta-D-glucuronic | Biosynth International Inc. |
| acid, sodium salt trihydrate (BCIG) | |
| O-Methyl-beta-D-glucuronide | Biosynth International Inc. |
| Isopropyl-beta-D-thiogalactopyranoside | Biosynth International Inc. |
| Lactose | Alpha Biosciences, Baltimore, MD |
| Casein Digest Peptone | Alpha Biosciences, Baltimore, MD |
| Monopotassium Phosphate | Becton, Dickinson and Company |
| Disodium Phosphate | MilliporeSigma Company |
| Sodium Dodecyl Sulfate | TCI America, Portland, OR |
| Methanol | MilliporeSigma Company |

TABLE 2
Nutrient Coating Composition

| | Amount (grams) |
|---|---|
| Components Added in Step 1 | |
| Casein Digest Peptone | 20 |
| Lactose | 5 |
| Sodium Chloride | 5 |
| Monopotassium Phosphate | 2.75 |
| Disodium Phosphate | 2.75 |
| Indicator Components Added in Step 2 | |
| 5-bromo-4-chloro-3-beta-D-glucuronide | 0.3 |
| O-Methyl-beta-D-glucuronide | 0.154 |
| 6-Chloro-3-indoxyl-beta-D-galactoside | 0.3 |
| Isopropyl-beta-D-thiogalactopyranoside | 0.4 |

### Preparatory Example 1. Incubation and Inoculation

The bacterial strains Escherichia coli (ATCC 51813) and Klebsiella oxytoca (ATCC 51817) were obtained from MICROBIOLOGICS, Incorporated (St. Cloud, Minn.) and individually incubated overnight in tryptic soy broth (TSB) at 37° C. and 200 rpm in an INNOVA44 incubator (New Brunswick Scientific, Enfield, Conn.). Each inoculum was prepared by serially diluting a single culture sample with Butterfield's Buffer. Each culture sample was diluted to yield a final concentration of about 10-250 colony forming unit (cfu) counts per 1 mL of inoculum.

### Preparatory Example 2. Microstructured Film Preparation

A polypropylene microstructured film having a plurality of conical post microstructures as shown in FIG. 3 was prepared according to the molding procedure described in Example 1 of United States Patent Application No. 20190001326A1 (incorporated herein by reference in its entirety). The conical post microstructures had the following dimensions: height (H2) of about 400 micrometers, width at base (W1) of about 180 micrometers, width at tip (W2) of about 150 micrometers. The conical posts had generally flat surfaces at the tip. The posts were arranged in a symmetrical pattern of rows with the center-to-center spacing of posts within a row being 429 micrometers. The posts in adjacent rows were offset by half a spacing so that the closest distance (center-to-center) between posts of adjacent rows was 479 micrometers.

### Preparatory Example 3. Plasma Treatment of Micro Structured Film

A silicon containing film layer [methods of forming described in U.S. Pat. No. 6,696,157 (David) and U.S. Pat. No. 8,664,323 (Iyer) and US Patent Application 2013/0229378 (Iyer)] was applied to the microstructured film of Preparatory Example 2 using a parallel plate capacitively coupled plasma reactor. The reactor chamber had a central cylindrical powered electrode with a surface area of 18.3 ft². After placing the micro-structured film on the powered electrode, the reactor chamber was pumped down to a base pressure of less than 1.3 Pa. A mixture of HMDSO (hexamethyldisiloxane) and oxygen gas was flowed into the chamber, at rates of 200 SCCM (standard cubic centimeters per minute) for HMDSO and 1000 SCCM oxygen. Treatment was carried out using a plasma enhanced chemical vapor deposition (CVD) method by coupling RF power into the reactor at a frequency of 13.56 MHz and an applied power of 7000 watts. Treatment time was controlled by moving the microstructured film through the reaction zone at rate of 30 feet/minute (9.14 meters/minute), resulting in an approximate exposure time of 10 seconds. After completing the deposition, RF power was turned off and gases were evacuated from the reactor. Following the first treatment, a second plasma treatment was carried out in the same reactor without returning the chamber to atmospheric pressure. Oxygen gas was flowed into the chamber at a rate of 1500 SCCM and 13.56 MHz RF power was subsequently coupled into the reactor with an applied power of 6000 watts. The microstructured film was then carried through the reaction zone at a rate of 30 feet/minute (9.14 meters/minute), resulting in an approximate exposure time of 10 seconds. At the end of this treatment time, the RF power and the gas supply were stopped and the chamber was returned to atmospheric pressure.

### Preparatory Example 4. Plasma Treatment of BOPP Film

A clear, biaxially-oriented polypropylene (BOPP) film (1.6 mil (0.04 mm) thick) without a microstructured surface was treated on both sides using the plasma treatment procedure described in Preparatory Example 3.

### Example 1. Microbial Detection Device

Microbial detection devices according to the device of FIG. 1 were constructed. For each device, the cover sheet was a clear, biaxially-oriented polypropylene (BOPP) film (1.6 mil (0.04 mm) thick) cut into a 76 mm wide by 102 mm long section. A circular section (5.1 cm diameter) of microstructured film (prepared according to Preparatory Examples 2 and 3) was applied so that the non-microreplicated surface of the film was adhesively laminated to the center of the cover sheet surface.

The substrate of the device was a clear, 3.0 mil (0.076 mm) PET film (Melanex 454, obtained from TEKRA LLC., New Berlin, Wis.) that was sequentially coated on one side with the microbial growth nutrient composition described in Table 2, an adhesive composition, and a guar gum composition according to the following procedure.

The microbial growth nutrient coating composition was prepared by vigorously mixing (using an air-driven overhead mixer with a JIFFY-type mixing impeller) the indicated amount of each component in Step 1 (Table 2) and 500 mL of purified water [obtained from a MILLI-Q Gradient Water Purification System (model #ZMQS6V00Y, Merck Millipore Corporation, Billerica, Mass.)] until the components were completely dissolved. The resulting solution had a pH of 6.8 (FE20 FIVEEASY pH Meter, Mettler-Toledo LLC, Columbus, Ohio). To this intermediate nutrient solution, a second solution consisting of the components in Step 2 (Table 2) dissolved in 10 mL of methanol was added. Next, guar gum (10 g) was added and vigorous stirring (750 rpm) was continued for about 10 minutes. Finally, while stirring slowly (450 rpm), 0.5 mL of 20% sodium dodecyl sulfate (aqueous) was added. The resulting solution was knife-coated onto one side of the PET substrate film with a 14 mil (0.35 mm) gap setting. The nutrient coated film was dried in an oven at 85° C. for 12 minutes to provide a dry coat weight of about 401 mg/24 in² (2.6 mg/cm²).

An isooctyl acrylate/acrylic acid (98/2 weight ratio) pressure-sensitive adhesive (PSA) coating formulation containing TTC (2,3,5-triphenyl tetrazolium chloride) indicator as described in Example 4 of U.S. Pat. No. 5,409,838 (which is incorporated herein by reference) was knife-coated onto the exposed nutrient coating with a 2 mil (0.05 mm) gap setting. The resulting coated film was dried in an oven at 65° C. for 6 minutes to provide a PSA coating having a dry coat weight of about 237 mg/24 in² (1.53 mg/cm²). The adhesive coated side of the substrate film was then powder coated with guar gum. The powder was evenly applied and excess powder was removed from the adhesive layer by hand shaking of the film followed by lightly brushing the surface with a paper towel. The final coat weight of the guar gum was about 600 mg/24 in² (3.88 mg/cm²).

The coated substrate was then cut to match the dimensions of the cover sheet (76 mm wide by 102 mm long). The finished devices were assembled by attaching a cover sheet to substrate (in a hinge-like fashion) along one edge (the 76 mm edge) of the substrate using double sided adhesive tape. For each device, the cover sheet and the substrate were oriented so that the coated surface of the substrate faced the microstructured surface on the cover sheet.

### Example 2. Microbial Detection Device

Microbial detection devices according to the device of FIG. 2 were constructed. For each device, the substrate was a clear, biaxially-oriented polypropylene (BOPP) film (1.6 mil (0.04 mm) thick) cut into a 76 mm wide by 102 mm long section. A circular section (5.1 cm diameter) of microstructured film (prepared according to Examples 2 and 3) was applied so that the non-microreplicated surface of the film was adhesively laminated to the center of the substrate surface.

The cover sheet of the device was a clear, 3.0 mil (0.076 mm) PET film (Melanex 454, obtained from TEKRA LLC.) that was sequentially coated on one side with the microbial growth nutrient composition described in Table 2, an adhesive composition, and a guar gum composition according to the following procedure.

The microbial growth nutrient coating composition was prepared by vigorously mixing (using an air-driven overhead mixer with a JIFFY-type mixing impeller) the indicated amount of each component in Step 1 (Table 2) and 500 mL of purified water [obtained from a MILLI-Q Gradient Water Purification System (model #ZMQS6V00Y, Merck Millipore Corporation)] until the components were completely dissolved. The resulting solution had a pH of 6.8 (FE20 FIVEEASY pH Meter). To this intermediate nutrient solution, a second solution consisting of the components in Step 2 (Table 2) dissolved in 10 mL of methanol was added. Next, guar gum (10 g) was added and vigorous stirring (750 rpm) was continued for about 10 minutes. Finally, while stirring slowly (450 rpm), 0.5 mL of 20% sodium dodecyl sulfate (aqueous) was added. The resulting solution was knife-coated onto one side of the PET cover sheet film with a 14 mil (0.35 mm) gap setting. The nutrient coated film was dried in an oven at 85° C. for 12 minutes to provide a dry coat weight of about 401 mg/24 in² (2.6 mg/cm²).

An isooctyl acrylate/acrylic acid (98/2 weight ratio) pressure-sensitive adhesive (PSA) coating formulation containing TTC (2,3,5-triphenyl tetrazolium chloride) indicator as described in Example 4 of U.S. Pat. No. 5,409,838 (which is incorporated herein by reference) was knife-coated onto the exposed nutrient coating with a 2 mil (0.05 mm) gap setting. The resulting coated film was dried in an oven at 65° C. for 6 minutes to provide a PSA coating having a dry coat weight of about 237 mg/24 in² (1.53 mg/cm²). The adhesive coated side of the cover sheet film was then powder coated with guar gum. The powder was evenly applied and excess powder was removed from the adhesive layer by hand shaking of the film followed by lightly brushing the surface with a paper towel. The final coat weight of the guar gum was about 600 mg/24 in² (3.88 mg/cm²).

The coated cover sheet was then cut to match the dimensions of the substrate (76 mm wide by 102 mm long). The finished devices were assembled by attaching a cover sheet to substrate (in a hinge-like fashion) along one edge (the 76 mm edge) of the substrate using double sided adhesive tape. For each device, the cover sheet and the substrate were oriented so that the coated surface of the cover sheet faced microstructured surface on the substrate.

### Comparative Example A

Microbial detection devices were constructed according to the procedure described in Example 1 with the exception that the circular section of microstructured film was replaced with a circular section of BOPP film (5.1 cm diameter) prepared according to Preparatory Example 4.

### Comparative Example B

Microbial detection devices were constructed according to the procedure described in Example 2 with the exception that the circular section of microstructured film was replaced with a circular section of BOPP film (5.1 cm diameter) prepared according to Preparatory Example 4.

### Example 3. Colony Count Using Device of Example 1

Finished detection devices of Example 1 were inoculated with an inoculum of either E. coli or K. oxytoca. The devices were placed on a flat, horizontal surface. The cover sheet of each device was lifted and 1 mL of inoculum (i.e., final dilution as described above) was added by pipette as a single application to a compact region in the center of the coated substrate. The cover sheet was gently returned to its original position so that the center of the microstructured surface contacted the added inoculum. In all of the devices, the inoculum sample spread such that the guar gum was evenly wetted and formed a hydrogel that filled the spaces between the posts of the microstructured film. The inoculum sample spread evenly across the area defined by the microstructured film and did not spread substantially beyond the outer edge of the microstructured film. Each device was incubated at 37° C. for 24 hours. At the end of the incubation period, the devices were evaluated for bacteria colony formation. Blue colored, punctate colonies were observed disposed across the surface of the hydrogel of each device inoculated with E. coli. Red colored, punctate colonies were observed disposed across the surface of the hydrogel of each device inoculated with K. oxytoca. The colonies were counted by visual examination. The results are presented in Table 3 as the mean colony count (cfu/mL) from two trials (n=2). As a control, the inocula were also analyzed using 3M PETRIFILM E. coli/Coliform Count Plates (obtained from the 3M Corporation, Maplewood, Minn.) according to the manufacturer's instructions. The mean colony counts (cfu/mL) using the control plates (n=2) are reported in Table 3.

### Example 4. Colony Counts Using Devices of Example 2

Finished detection devices of Example 2 were inoculated with an inoculum of either E. coli or K. oxytoca. The devices were placed on a flat, horizontal surface. The cover sheet of each device was lifted, and 1 mL of inoculum (i.e., final dilution as described above) was added by pipette as a single application to the center portion of the microstructured film. The inoculum sample spread evenly across the entire microstructured surface and did not spread beyond the outer edge of the microstructured film. The coated cover sheet was gently returned to its original position. In all of the devices, the guar gum was evenly wetted forming a hydrogel filled the spaces between the posts of the microstructured film. The hydrogel did not spread substantially beyond the outer edge of the microstructured film. Each device was incubated at 37° C. for 24 hours. At the end of the incubation period, the devices were evaluated for bacteria colony formation. Blue colored, punctate colonies were observed disposed across the surface of the hydrogel of each device inoculated with E. coli. Red colored, punctate colonies were observed disposed across the surface of the hydrogel of each device inoculated with K. oxytoca. The colonies were counted by visual examination. The results are presented in Table 3 as the mean colony count (cfu/mL) from two trials (n=2).

### Comparative Example C. Colony Counts Using Devices of Comparative Example A

Finished detection devices of Comparative Example A were inoculated with an inoculum of either E. coli or K. oxytoca. The devices were placed on a flat, horizontal surface. The cover sheet of each device was lifted, and 1 mL of inoculum (i.e., final dilution as described above) was added by pipette as a single application to a compact region in the center of the coated substrate. The cover sheet was gently returned to its original position so that the center of the plasma treated BOPP film section contacted the added inoculum. The inoculum spread in a random and irreproducible manner. In some devices, the inoculum sample spread in an irregular pattern that wetted areas of the substrate significantly beyond the area defined by the circular plasma treated BOPP film section of the cover sheet. In other devices, the inoculum sample spread in an irregular pattern that wetted only a portion of the substrate area defined by (i.e. underlying) the circular plasma treated BOPP film section of the cover sheet. Each device was incubated at 37° C. for 24 hours. At the end of the incubation period, the devices were evaluated for bacteria colony formation. Blue colored, punctate colonies were observed disposed across the surface of the hydrogel of each device inoculated with E. coli. Red colored, punctate colonies were observed disposed across the surface of the hydrogel of each device inoculated with K. oxytoca. The colonies were counted by visual examination. The results are presented in Table 3 as the mean colony count (cfu/mL) from two trials (n=2).

### Comparative Example D. Colony Counts Using Devices of Comparative Example B

Finished detection devices of Comparative Example B were inoculated with an inoculum of either E. coli or K. oxytoca. The devices were placed on a flat, horizontal surface. The cover sheet of each device was lifted, and 1 mL of inoculum (i.e., final dilution as described above) was added by pipette as a single application to the center portion of the plasma treated BOPP film section of the substrate. The coated cover sheet was gently returned to its original position. The inoculum spread in a random and irreproducible manner. In some devices, the inoculum sample spread in an irregular pattern that wetted areas of the cover sheet significantly beyond the area defined by the circular plasma treated BOPP film section of the substrate. In other devices, the inoculum sample spread in an irregular pattern that wetted only a portion of the cover sheet area defined by (i.e. overlying) the circular plasma treated BOPP film section of the substrate. Each device was incubated at 37° C. for 24 hours. At the end of the incubation period, the devices were evaluated for bacteria colony formation. Blue colored, punctate colonies were observed disposed across the surface of the hydrogel of each device inoculated with E. coli. Red colored, punctate colonies were observed disposed across the surface of the hydrogel of each device inoculated with K. oxytoca. The colonies were counted by visual examination. The results are presented in Table 3 as the mean colony count (cfu/mL) from two trials (n=2).

TABLE 3

Colony Count (cfu/mL)

| Device Description | E. coli | K. oxytoca |
|---|---|---|
| Example 1 | 14 | 10 |
| Example 2 | 14 | 16 |
| Comparative Example A | 16 | 12 |
| Comparative Example B | 15 | 14 |
| 3M PETRIFILM E. coli/Coliform | 15 | 11 |
| Plate (Control Device) | | |

### Example 5. Leak Testing of Devices of Example 1

Eight finished detection devices of Example 1 were tested for sample leaking using a solution of methylene blue (0.1 mg/mL) in deionized water as the inoculation sample. The blue color of the solution was used to aid in visualizing leakage of the added sample. The devices were placed on a flat, horizontal surface. The cover sheet of each device was lifted, and 1 mL of the methylene blue solution was added by pipette as a single application to a compact region in the center of the coated substrate. The cover sheet was gently returned to its original position and the device was maintained on the horizontal surface without being moved for 30 seconds. The device was then grasped by hand at the hinged edge, moved to a vertical orientation in a position above the horizontal surface, and held steady for 15 seconds. During the test, no liquid was observed to leak out of any of the devices.

Comparative Example E. Leak Testing of Devices of Comparative Example A Eight finished detection devices of Comparative Example A were tested for sample leaking according to the procedure described in Example 5. During the test, liquid was observed to leak out of all of the devices. For five devices, the liquid leaked out of the device immediately (<1 second) after moving the device to the vertical orientation. For three devices, the liquid leaked out of the devices 2-5 seconds after moving the device to the vertical position.

### Example 6. Leak Testing of Devices of Example 2

Eight finished detection devices of Example 2 were tested for sample leaking using a solution of methylene blue (0.1 mg/mL) in deionized water as the inoculation sample. The devices were placed on a flat, horizontal surface. The cover sheet of each device was lifted, and 1 mL of the methylene blue solution was added by pipette as a single application to the center portion of the microstructured film. The cover sheet was gently returned to its original position and the device was maintained on the horizontal surface without being moved for 30 seconds. The device was then grasped by hand at the hinged edge, moved to a vertical orientation in a position above the horizontal surface, and held steady for 15 seconds. During the test, no liquid was observed to leak out of any of the devices.

### Comparative Example F. Leak Testing of Devices of Comparative Example B

Eight finished detection devices of Comparative Example B were tested for sample leaking using a solution of methylene blue (2 mg/mL) in deionized water as the inoculation sample. The devices were placed on a flat, horizontal surface. The cover sheet of each device was lifted, and 1 mL of the methylene blue solution added by pipette as a single application to the center portion of the plasma treated BOPP film section of the substrate. The cover sheet was gently returned to its original position and the device was maintained on the horizontal surface without being moved for 30 seconds. The device was then grasped by hand at the hinged edge, moved to a vertical orientation in a position above the horizontal surface, and held steady for 15 seconds. For three devices, the liquid leaked out of the device immediately (<1 second) after moving the device to the vertical position. For four devices, the liquid leaked out of the devices 3-5 seconds after moving the device to the vertical position. For one device, the liquid migrated in the device, but did not leak out of the device.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A device for growing microorganisms, comprising:
a self-supporting, water-proof substrate having an inner-facing surface and an outer-facing surface;
a cover sheet having an inner-facing surface and an outer-facing surface, the cover sheet adhered to at least a portion of the substrate;
a microstructured surface on the inner-facing surface of the coversheet, wherein the microstructured surface comprises a plurality of microstructures; and
a substantially dry, gelling agent and a substantially dry, microbial growth nutrient composition disposed on the microstructured surface of the microstructured film or on a portion of the inner-facing surface of the substrate.

2. A device for growing microorganisms, comprising:
a self-supporting, water-proof substrate having an inner-facing surface and an outer-facing surface;
a cover sheet having an inner-facing surface and an outer-facing surface, the cover sheet adhered to at least a portion of the substrate;
a microstructured surface on the inner-facing surface of the substrate, wherein the microstructured surface comprises a plurality of microstructures; a substantially dry, gelling agent; and
a substantially dry, microbial growth nutrient composition disposed on the microstructured surface of the microstructured film or on a portion of the inner-facing surface of the cover sheet.

3. The device of claim 1, wherein the plurality of micro structures are orderly arranged on the microstructured surface.

4. The device of claim 1, wherein the plurality of micro structures are randomly arranged on the microstructured surface.

5. The device of claim 1, wherein the microstructures have an aspect ratio from 1:1 to 200:1.

6. The device of claim 1, wherein the microstructured surface covers a part of the inner-facing surface of the coversheet or substrate.

7. The device of claim 1, wherein the microstructured surface covers the whole inner-facing surface of the coversheet or substrate.

8. The device of claim 1, wherein the microstructured surface covers the whole inner-facing surface of the coversheet or substrate.

9. The device of claim 1, wherein the microstructured surface has a circular cross-section.

10. The device of claim 1, wherein the cover sheet comprises a transparent film.

11. The device of claim 10, wherein the transparent film is selected from the group consisting of polyester, polyethylene, polypropylene, polystyrene and silicone.

12. The device of claim 1, further comprising an adhesive layer to cover the gelling agent or growth nutrient composition.

13. The device of claim 1, wherein the microstructured surface is part of a microstructured film.

14. A method comprising:
providing a device according to claim 1;
adding a predetermined volume of a sample containing at least one microorganism onto the self-supporting, water-proof substrate or the microstructured surface of the microstructured film to form an inoculated device;
contacting the cover sheet to the self-supporting, water-proof substrate;
incubating the inoculated device; and
detecting the presence or an absence of a colony of the target microorganism in the device.

15. The method of claim 14, further comprising forming a hydrogel of growth nutrient composition and the hydrogel partially fills the spaces between the plurality of microstructures.
